(19) United States
(12) Patent Application Publication
(10) Pub. No.: US 2024/0216042 A1
(43) Pub. Date: Jul. 4, 2024

(54) MEDICAL DEVICE WITH A BILATERAL JAW CONFIGURATION FOR NERVE STIMULATION

(71) Applicant: CILAG GMBH INTERNATIONAL, ZUG (CH)

(72) Inventors: David C. Yates, Morrow, OH (US); Jeffrey D. Messerly, Cincinnati, OH (US); Cameron D. McLain, Fairfield, OH (US); Peter K. Shires, Robbinsville, NC (US); Frederick L. Estera, Cincinnati, OH (US); Cameron R. Nott, Fairfield, OH (US); Foster B. Stulen, Johns Island, SC (US); Christopher A. Papa, Cincinnati, OH (US)

(21) Appl. No.: 18/408,513

(22) Filed: Jan. 9, 2024

Related U.S. Application Data
(63) Continuation of application No. 16/574,843, filed on Sep. 18, 2019, now Pat. No. 11,864,820.
(63) Continuation of application No. 15/493,122, filed on Apr. 20, 2017, now Pat. No. 10,456,193.
(60) Provisional application No. 62/331,123, filed on May 3, 2016.

(51) Int. Cl. A61B 18/14; A61B 17/00; A61B 17/28; A61B 17/285; A61B 17/29; A61B 17/295; A61B 17/32; A61B 18/00; A61B 18/12; A61B 18/16; A61N 1/04; A61N 1/05; A61N 1/36

(52) U.S. Cl. CPC A61B 18/1442; A61B 17/2812; A61B 17/285; A61B 17/29; A61B 17/295; A61B 17/320068; A61B 17/320092; A61N 1/0551; A61N 1/3605; A61N 1/36057; A61N 1/36146; A61B 2017/00017; A61B 2017/2945; A61B 2017/320093; A61B 2017/320094; A61B 2017/320095; A61B 2018/00083; A61B 2018/00434; A61B 2018/00601; A61B 2018/00607; A61B 2018/0063; A61B 2018/00958; A61B 2018/00994; A61B 2018/1253; A61B 2018/126; A61B 2018/162; A61N 1/04

(57) ABSTRACT

Aspects of the present disclosure are presented for a single surgical instrument configured to grasp, seal, and/or cut tissue through application of therapeutic energy, and also detect nerves through application of non-therapeutic electrical energy. A medical device may include two jaws at an end effector, used to apply therapeutic energy and to perform surgical procedures. The therapeutic energy may be in the form of ultrasonic vibrations or higher voltage electrosurgical energy. One of the jaws may be configured to cut tissue through application of the blade. In addition, one or both of the two jaws may be configured to apply nontherapeutic energy for nerve stimulation probing. The application of therapeutic energy may be disabled while the nontherapeutic nerve stimulation energy is applied, and vice versa. The nontherapeutic nerve stimulation energy may be applied to the use of one or more probes positioned near one or both of the jaws.

This application is a continuation application claiming priority under 35 U.S.C. § 120 to U.S. patent application Ser. No. 16/574,843, entitled “MEDICAL DEVICE WITH A BILATERAL JAW CONFIGURATION FOR NERVE STIMULATION,” now U.S. Pat. No. 11,864,820, which was filed on Sep. 18, 2019 and which claims priority under 35 U.S.C. § 120 to U.S. patent application Ser. No. 15/493,122, entitled “MEDICAL DEVICE WITH A BILATERAL JAW CONFIGURATION FOR NERVE STIMULATION,” now U.S. Pat. No. 10,456,193, which was filed on Apr. 20, 2017 and which claims the benefit under 35 U.S.C. § 119(e) of U.S. Provisional Patent Application Ser. No. 62/331,123, entitled “MEDICAL DEVICE WITH A BILATERAL JAW CONFIGURATION FOR NERVE STIMULATION,” which was filed on May 3, 2016, filed May 3, 2016, the entire disclosures of each of which are hereby incorporated by reference herein.

### TECHNICAL FIELD

The present disclosure is related generally to medical devices with various mechanisms for grasping, cutting, and sealing tissue. In particular, the present disclosure is related to medical devices with grasping instruments that perform nerve stimulation using non-therapeutic energy in one or more probes in the same device.

### BACKGROUND

During surgery, energy used to cut and seal tissue, such as ultrasonic vibrations or electrosurgical energy, may be damaging to certain body tissue that is not intended to be operated on though it be present in a surgical site. For example, nerves may be damaged that come into contact with energy applied at surgical and therapeutic levels. On the other hand, low levels of energy applied onto or close to nerves (e.g., ˜2 mm) may cause a visible vibration of the associated muscles to alert a surgeon to their presence. This visible indication may provide the surgeon enough information to safely perform the surgery while avoiding delicate nerve endings. Traditionally, surgeons may therefore utilize multiple devices to perform the surgery, such as one device to detect nerves by applying lower voltage to nerve stimulation probes, and another device to grasp, seal, and/or cut tissue by applying ultrasonic or higher voltage therapeutic energy. It may be desirable to utilize a surgical tool that applies therapeutic energy, e.g., ultrasonic or higher voltage electrosurgical energy, and also includes nerve sensing capabilities, such as carefully positioned lower voltage nerve stimulation probes, in a single device to allow for safer and more efficient surgical procedures.

While several devices have been made and used, it is believed that no one prior to the inventors has made or used the device described in the appended claims.

### BRIEF SUMMARY

In some aspects, a surgical instrument is provided.

In one example, a surgical instrument is presented comprising: a first jaw handle and a second jaw handle pivotally coupled about a joint; the first jaw handle comprising a first jaw at a distal end of the first jaw handle; the second jaw handle comprising a second jaw at a distal end of the second jaw handle; the first and second jaws configured to grasp tissue therebetween when the first and second jaw handles are pivotally rotated about the joint into a closed position; at least one of the first and second jaws configured to deliver therapeutic energy at a higher energy level for sealing or cutting tissue at a surgical site; and at least one of the first and second jaws configured to deliver nontherapeutic energy at a lower energy level for providing electrical stimulation to nerve tissue at the surgical site.

In another example of the surgical instrument in example 1, the therapeutic energy comprises ultrasonic energy.

In another example of the surgical instrument in example 1, the therapeutic energy comprises high frequency electrosurgical energy.

In another example of the surgical instrument in example 1, both the first and second jaws are configured to deliver the nontherapeutic energy to the tissue at the surgical site, and wherein the nontherapeutic energy delivered at both the first and second jaws are at the same electrical potential.

In another example of the surgical instrument in example 1, at least one of the first and second jaws comprises an insulating material on an inside portion of said jaw such that the first jaw contacts the second jaw in the closed position by way of touching the insulating material.

In another example, a surgical instrument is presented comprising: a handle assembly; and an end effector communicatively coupled to the handle assembly and comprising a first and second jaw, the handle assembly configured to manipulate the first and second jaws to define an open position and a closed position; and the end effector configured to: deliver therapeutic energy at a higher energy level for sealing or cutting tissue at a surgical site; and deliver nontherapeutic energy at a lower energy level for providing electrical stimulation to nerve tissue at the surgical site.

In another example of the surgical instrument in example 6, the end effector further comprises an electrical stimulation probe configured to deliver the nontherapeutic energy to stimulate the nerve tissue at the surgical site.

In another example of the surgical instrument in example 7, the electrical stimulation probe is retractable and is further configured to retract in a proximal direction toward the handle assembly, and to protract in a distal direction away from the handle assembly.

In another example of the surgical instrument in example 7, the end effector further comprises a return probe, wherein the electrical stimulation probe and the return probe complete a circuit when both the return probe and the electrical stimulation probe contact tissue at the surgical site, and wherein the return probe acts as an electrical return for the nontherapeutic energy.

In another example of the surgical instrument in example 7, the electrical stimulation probe is configured to contour substantially near a first side of the end effector, the first side of the end effector including a first portion of a distal tip of the end effector defined by the first and second jaws.

In another example of the surgical instrument in example 10, the end effector further comprises a return probe configured to contour substantially near a second side of the end effector opposite to the first side, the second side of the end effector including a second portion of the distal tip of the end effector defined by the first and second jaws, wherein the electrical stimulation probe and the return probe complete a circuit when both the return probe and the electrical stimulation probe contact tissue at the surgical site.

In another example of the surgical instrument in example 6, the end effector further comprises a first electrical protrusion probe coupled to a distal end of the first or second jaw and configured to deliver the nontherapeutic energy to stimulate the nerve tissue at the surgical site.

In another example of the surgical instrument in example 12, wherein the end effector further comprises a second electrical protrusion return probe coupled to the distal end of the first or second jaw and configured to complete a circuit with the first electrical protrusion when both the first and second probes contact tissue at the surgical site.

In another example of the surgical instrument in example 6, the surgical instrument further comprises a shaft coupled to a distal end of the handle assembly and a proximal end of the end effector, the shaft configured to electrically couple the end effector to the handle assembly.

In another example, a surgical system is presented comprising: a handle assembly; a power generator electrically coupled to the handle assembly; and an end effector communicatively coupled to the handle assembly and electrically coupled to the power generator and comprising a first and second jaw, the handle assembly configured to manipulate the first and second jaws to define an open position and a closed position; the end effector configured to: selectively deliver therapeutic energy at a higher energy level for sealing or cutting tissue at a surgical site; and selectively deliver nontherapeutic energy at a lower energy level for providing electrical stimulation to nerve tissue at the surgical site; the power generator configured to: generate therapeutic energy at the higher energy level to be transmitted to the end effector; and generate nontherapeutic energy at the lower energy level to be transmitted to the end effector.

In another example of the surgical system in example 15, the end effector further comprises an electrical stimulation probe configured to deliver the nontherapeutic energy to stimulate the nerve tissue at the surgical site.

In another example of the surgical system in example 16, the electrical stimulation probe is retractable and is further configured to retract in a proximal direction toward the handle assembly, and to protract in a distal direction away from the handle assembly.

In another example of the surgical system in example 16, the end effector further comprises a return probe, wherein the electrical stimulation probe and the return probe complete a circuit when both the return probe and the electrical stimulation probe contact tissue at the surgical site.

In another example of the surgical system in example 16, the electrical stimulation probe is configured to contour substantially near a first side of the end effector, the first side of the end effector including a first portion of a distal tip of the end effector defined by the first and second jaws.

In another example of the surgical system in example 15, the end effector further comprises a return probe configured to contour substantially near a second side of the end effector opposite to the first side, the second side of the end effector including a second portion of the distal tip of the end effector defined by the first and second jaws, wherein the electrical stimulation probe and the return probe complete a circuit when both the return probe and the electrical stimulation probe contact tissue at the surgical site.

In another example of the surgical system in example 18, the therapeutic energy is monopolar and the return probe further acts as an electrical return for the therapeutic energy.

The foregoing summary is illustrative only and is not intended to be in any way limiting. In addition to the illustrative aspects and features described above, further aspects and features will become apparent by reference to the drawings and the following detailed description.

### BRIEF DESCRIPTION OF THE DRAWINGS

The novel features of the aspects described herein are set forth with particularity in the appended claims. These aspects, however, both as to organization and methods of operation may be better understood by reference to the following description, taken in conjunction with the accompanying drawings as follows.

FIG. 1 illustrates a medical device configurable with both nerve stimulation and therapeutic functions, according to various aspects.

FIG. 2 illustrates an alternative design of a surgical instrument configured to deliver therapeutic and nontherapeutic energy to a surgical site, according to some aspects.

FIG. 3 shows an alternative design for including a nerve stimulation probe alongside the jaws of the surgical instrument, according to some aspects.

FIG. 4 shows a side view of the retractable probes, shown in FIG. 3.

FIG. 5 shows portions of a retraction mechanism at the handle assembly to manipulate the retractable probes of FIGS. 3 and 4, according to some aspects.

FIG. 6 shows another alternative design for including a nerve stimulation probe alongside the contours of the jaws of the surgical instrument, according to some aspects.

FIG. 7 shows the side view of the perimeter probes configuration of FIG. 6, according to some aspects.

FIG. 8 shows yet another alternative design for including nerve stimulation at the jaws, including an electrical protrusion probe and return protrusion probe, according to some aspects.

FIG. 9 shows yet another alternative design for including nerve stimulation in a surgical device, including enabling the blade to supply both therapeutic and nontherapeutic energy, according to some aspects.

FIG. 10 is a block diagram of a surgical system comprising a grasping instrument with motor-driven components to apply energy to a surgical site, the surgical instrument coupled to a generator wired stimulating source (or alternatively having an internal generator), according to some aspects.

FIG. 11 illustrates a button based on a capacitive switch, according to some aspects.

FIG. 12 illustrates one aspect of a nerve stimulation system 1200, according to some aspects.

### DETAILED DESCRIPTION

In the following detailed description, reference is made to the accompanying drawings, which form a part hereof In the drawings, similar symbols and reference characters typically identify similar components throughout the several views, unless context dictates otherwise. The illustrative aspects described in the detailed description, drawings, and claims are not meant to be limiting. Other aspects may be utilized, and other changes may be made, without departing from the scope of the subject matter presented here.

The following description of certain examples of the technology should not be used to limit its scope. Other examples, features, aspects, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, aspects, examples, etc., described herein may be combined with any one or more of the other teachings, expressions, aspects, examples, etc., that are described herein. The following-described teachings, expressions, aspects, examples, etc., should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Also, in the following description, it is to be understood that terms such as front, back, inside, outside, top, bottom, and the like are words of convenience and are not to be construed as limiting terms. Terminology used herein is not meant to be limiting insofar as devices described herein, or portions thereof, may be attached or utilized in other orientations. The various aspects will be described in more detail with reference to the drawings. Throughout this disclosure, the term “proximal” is used to describe the side of a component, e.g., a shaft, a handle assembly, etc., closer to a user operating the surgical instrument, e.g., a surgeon, and the term “distal” is used to describe the side of the component further from the user operating the surgical instrument.

Aspects of the present disclosure are presented for a single surgical instrument configured to grasp, seal, and/or cut tissue through application of therapeutic energy, e.g., ultrasonic and/or higher voltage electrosurgical energy, and also detect nerves through application of non-therapeutic electrical energy. Conventionally, at least two medical devices may be used to perform the combination of these actions. However, visibility may be reduced by having multiple devices crowding the surgical site, making it possibly less safe than if a single device could perform all of the aforementioned functions. Furthermore, a surgeon's precision may be compromised if multiple instruments are alternated in and out of the surgical site to perform different functions repeatedly.

Various features described herein may be incorporated in electrosurgical devices for applying electrical energy to tissue for therapeutic purposes in order to treat and/or destroy the tissue are also finding increasingly widespread applications in surgical procedures. An electrosurgical device typically includes a hand piece, an instrument having a distally-mounted end effector (e.g., one or more electrodes). The end effector can be positioned against the tissue such that electrical current is introduced into the tissue. Electrosurgical devices can be configured for bipolar or monopolar operation. During bipolar operation, current is introduced into and returned from the tissue by active and return electrodes, respectively, of the end effector. During monopolar operation, current is introduced into the tissue by an active electrode of the end effector and returned through a return electrode (e.g., a grounding pad or dispersive electrode that is attached to or adjacent the patient at a location remote to the surgical site and electrically coupled to the patient capacitively or directly by way of a conductive adhesive) separately located on a patient's body. Heat generated by the current flowing through the tissue may form hemostatic seals within the tissue and/or between tissues and thus may be particularly useful for sealing blood vessels, for example. The end effector of an electrosurgical device may also include a cutting member that is movable relative to the tissue and the electrodes to transect the tissue.

In some aspects, a medical device includes two jaws at an end effector, used to apply therapeutic energy and to perform grasping, sealing, and/or cutting procedures. The end effector may be formed at the distal end of a shaft, the proximal end of the shaft communicatively coupled to a handle assembly. In other cases, the end effector may be formed at the end of a pair of clamp arms in a scissor-like configuration. One or both of the jaws may be configured to supply therapeutic energy, such as in the form of ultrasonic vibrations or higher voltage electrosurgical energy. In some aspects, one of the jaws may be configured to cut tissue through application of a blade. In addition, one or both of the two jaws may be configured to apply nontherapeutic energy for nerve stimulation probing, such as applying lower amplitude electrical energy. In some aspects, the application of therapeutic energy may be disabled while the nontherapeutic nerve stimulation energy is applied, and vice versa. In some aspects, the nontherapeutic nerve stimulation energy may be applied to the use of one or more probes positioned near one or both of the jaws.

Electrical energy applied by an electrosurgical device can be transmitted to the instrument by a generator in communication with the handle assembly. The electrical energy may be in the form of radio frequency (“RF”) energy. RF energy is a form of electrical energy that may be in the frequency range of 200 kilohertz (kHz) to 1 megahertz (MHz). In application, an electrosurgical device can transmit low frequency RF energy through tissue, which causes ionic agitation, or friction, in effect resistive heating, thereby increasing the temperature of the tissue. Because a sharp boundary is created between the affected tissue and the surrounding tissue, surgeons can operate with a high level of precision and control, without sacrificing un-targeted adjacent tissue. The low operating temperatures of RF energy is useful for removing, shrinking, or sculpting soft tissue while simultaneously sealing blood vessels. RF energy works particularly well on connective tissue, which is primarily comprised of collagen and shrinks when contacted by heat.

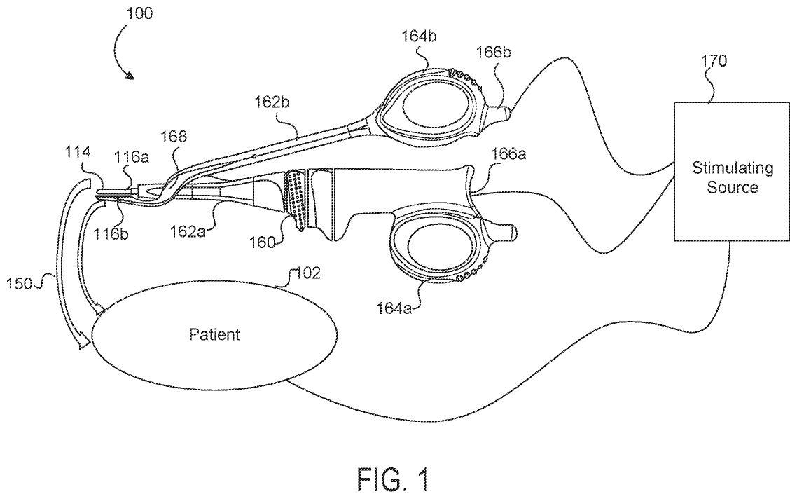

Referring to FIG. 1, a medical device 100 is illustrated, configurable with both nerve stimulation and therapeutic functions, according to various aspects. The medical device 100 comprises two jaws 116a and 116b at the distal end to define an end effector 114, both jaws separately connected to jaw arms 162a and 162b, respectively. Jaw handles 164a and 164b are coupled to the jaw arms 162a and 162, respectively, at the proximal ends. The jaws 116a and 116b may be pivotally connected at a joint 168, thereby allowing the jaws to clamp and/or grasp tissue between them. In some aspects, one or both of the jaws 116a and 116b may be configured to deliver therapeutic energy to the contact tissue through various means, such as ultrasonic energy, high frequency electrosurgical energy, resistive heating elements, microwaves, and so forth. In some aspects, an activation switch, roller, or button 160 may be included in one of the arms to adjust the degree of therapeutic energy.

In addition, the medical device 100 may be configured to deliver nontherapeutic, nerve stimulation probing energy at one or both of the jaws 116a and 160b. This energy may be formed at a low frequency or voltage so as to not damage any nerves or other tissues when the jaws 116a and 116b come into contact with tissue in this mode. In some aspects, both of the jaws may be configured to deliver the nontherapeutic probing energy to create a bilateral nerve stimulation configuration. A stimulating source 170 may be electrically coupled to the medical device 100, such as through the energy ports 166a and 166b, to supply the probing energy through the two jaw handles 164a and 164b. Both of the jaws may be receiving energy at the same electrical potential. The energy may then be applied to the patient 102 through the jaws 116a and 116b, as indicated by the arrows 150. The electrical stimulation path may be completed through use of, for example a subdermal needle probe, a patient grounding clip wire, or patient grounding pad. Thus, when in this configuration, the medical device 100 may be configured to detect when the jaws 116a and/or 116b approach any nerves at a surgical site. For example, application of this energy touching or nearly touching (e.g., ˜2 mm) the nerves may result in a visible vibration of the nerve, to indicate to the surgeon the presence of the nerve.

In some aspects, locating the nerves by this type of stimulation may be achieved through any of three desired usage modes. In one case, nerves may be queried through probing by the tips of the jaws 116a and 116b. That is, nerves or other non-muscle tissue may twitch or vibrate if the tips of the jaws 116a and/or 116b are on or near (e.g., ˜1-5 mm) them. In testing with about 0.5 mA stimulation output, nerves are typically stimulated when the conducting member (tips of the jaws 116a and/or 116b) is on or touching the nerve or touching tissue that is immediately adjacent (less than about 1 mm) to the nerve. Likewise, with a 2 mA stimulation output, nerves are typically stimulated when the conducting member touches adjacent tissue that is at about 2 to 5 mm from the nerve. A second case includes clamping of tissue to detect the nerves. At least one inside clapping surface of one of the jaws 116a or 116b is configured to apply the nerve stimulation energy. A third case includes detection through spread dissection. Spreading tissue by widening the jaws may detect nerves with at least one of the outside surfaces configured to be active to apply the nontherapeutic nerve stimulation energy. In other configurations, more than one of these usage modes may be implemented in the medical device 100.

In some aspects, the jaws 116a and 116b may be electrically isolated even when the jaws are in a closed position or are grasping tissue. Insulating pins may be placed on the inside of at least one of the jaws 116a or 116b, such that the jaws touch only at the insulating pins. In other cases, insulating bands or other non conductive materials may be positioned around one or both of the jaws 116a or 116b such that the jaws touch only at the insulating bands or other non conductive materials. In this way, sufficient physical space may be allocated to allow for ultrasonic vibratory isolation, or in other cases for electrical isolation as may be utilized with bipolar electrosurgery jaws.

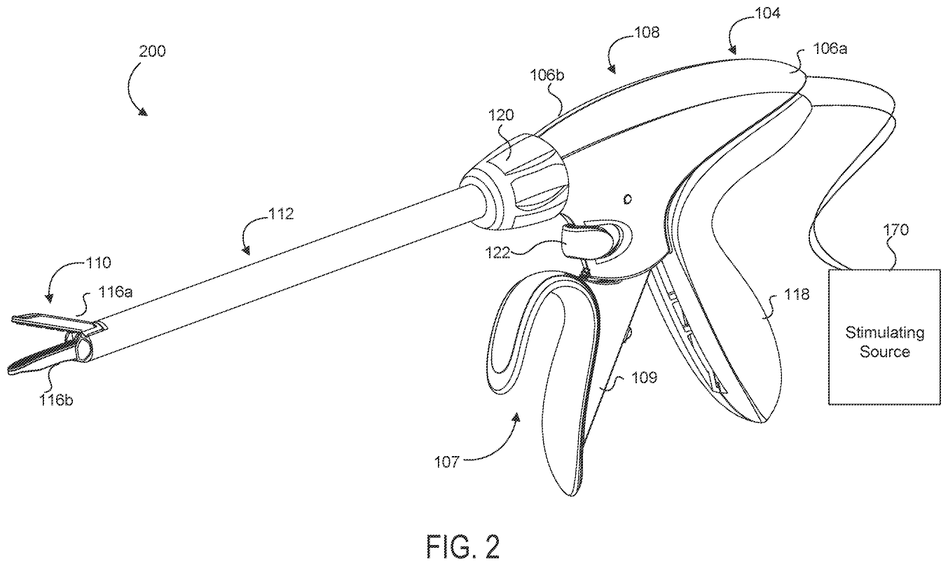

FIG. 2 illustrates an alternative design of a surgical instrument 200 configured to deliver therapeutic and nontherapeutic energy to a surgical site, according to some aspects. The instrument 200 may include a trigger assembly 107 and a closure system arrangement for closing the jaws 116a and 116b at the end effector 110 comprising a spring driven cam closure mechanism, according to some aspects. The spring driven cam closure system is configured to close the set of opposing jaws 116a, 116b, and in some cases fire a cutting element in the end effector 110. The trigger assembly 107 is configured to clamp and fire an end effector 110 coupled to the shaft assembly 112 of the surgical instrument 200. The surgical instrument 200 also includes a handle assembly 104, a shaft assembly 112, and the end effector 110. The shaft assembly 112 comprises a proximal end and a distal end. The proximal end of the shaft assembly 112 is coupled to the distal end of the handle assembly 104. The end effector 110 is coupled to the distal end of the shaft assembly 112. The handle assembly 104 comprises a pistol grip 118. The handle assembly 104 comprises a left handle housing shroud 106a and a right handle housing shroud 106b. The trigger assembly 107 comprises a trigger 109 actuatable towards the pistol grip 118. A rotatable shaft knob 120 is configured to rotate the shaft assembly 112 with respect to the handle assembly 104. The handle assembly 104 further comprises an energy button 122 configured to provide therapeutic electrosurgical energy to one or more electrodes in the end effector 110. The shaft assembly 112 comprises a closure/jaw actuator, a firing/cutting member actuator, and an outer sheath. In some aspects, the outer sheath comprises the closure actuator. The outer sheath comprises one or more contact electrodes on a distal end configured to interface with the end effector 110. The one or more contact electrodes are operatively coupled to the energy button 122, the tissue sealer mode selection assembly 108, and the stimulating source 170.

The energy source may be suitable for therapeutic tissue treatment, tissue cauterization/sealing, as well as sub-therapeutic treatment and measurement. The energy button 122 controls the delivery of energy to the electrodes. The device 200 also is configured to switch to applying non-therapeutic energy at the end effector 110, in some cases through a toggle switch or button, such as by pressing energy button 122 a second time to switch to nerve stimulation mode. In other cases, a separate button or switch activation, such as through rotating the knob 120 into a certain orientation, or pressing another button, not shown, may allow for the activation of the nerve stimulation energy. The stimulating source 170 may be configured to generate energy for one or both modes. Additional example details for the energy generation waveforms will be described in more detail, below.

It will be appreciated that the user may select a treatment or diagnostic output or the system may select a treatment or diagnostic output as appropriate to the situation.

As used throughout this disclosure, a button refers to a switch mechanism for controlling some aspect of a machine or a process. The buttons may be made out of a hard material such as usually plastic or metal. The surface may be formed or shaped to accommodate the human finger or hand, so as to be easily depressed or pushed. Buttons can be most often biased switches, even though many un-biased buttons (due to their physical nature) require a spring to return to their un-pushed state. Terms for the “pushing” of the button, may include press, depress, mash, and punch.

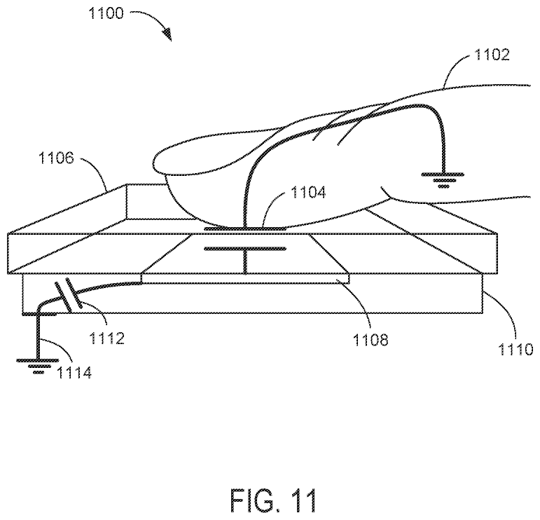

FIG. 11 illustrates a button based on a capacitive switch, according to some aspects of the present disclosure. In other aspects, the button could be based on optical interrupter technology. Returning to FIG. 11, a capacitive touch switch system 1100 is shown. The capacitive touch switch 1100 comprises an electrically insulative cover plate 1106 located above a printed circuit board substrate 1110. An electrically conductive pad 1108, usually formed of copper or other electrically conductive metal. The capacitive touch switch 1100 is formed when a finger 1102 touches the cover plate 1106 and a finger capacitance 1104 is developed between the finger 1102 and the electrically conductive pad 1108 located below the cover plate 1106. The finger capacitance 1104 is coupled to ground through the person's body. When the finger 1102 is in contact with the cover plate 1106, the finger capacitance 1104 is in parallel with the pad capacitance 1112 which is coupled to a ground 1114 located elsewhere in the system. The capacitive touch switch system 1100 is one example of how buttons employed in the example surgical devices may be implemented.

Referring back to FIG. 2, in some aspects, the end effector 110 is coupled to the distal end of the shaft assembly 112. The end effector 110 comprises a first jaw member 116a and a second jaw member 116b. The first jaw member 116a is pivotally coupled to the second jaw member 116b. The first jaw member 116a is pivotally moveable with respect to the second jaw member 116b to grasp tissue therebetween. In some aspects, the second jaw member 116b is fixed. In other aspects, the first jaw member 116a and the second jaw member 116b are pivotally movable. The end effector 110 comprises at least one electrode. The electrode is configured to deliver electrosurgical energy. Energy delivered by the electrode may comprise, for example, radiofrequency (RF) energy, sub-therapeutic RF energy, ultrasonic energy, and/or other suitable forms of energy. In some aspects, a cutting member (not shown) is receivable within a longitudinal slot defined by the first jaw member 116a and/or the second jaw member 116b. The cutting member is configured to cut tissue grasped between the first jaw member 116a and the second jaw member 116b. In some aspects, the cutting member comprises an electrode for delivering energy, such as, for example, RF and/or ultrasonic energy.

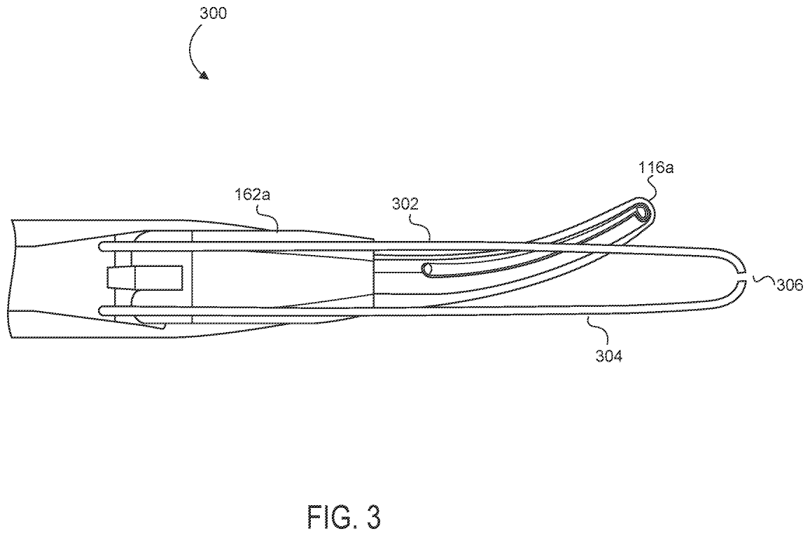

Referring to FIG. 3, illustration 300 shows an alternative design for including a nerve stimulation probe alongside jaws 116a and 116b, according to some aspects. Here, a retractable probe configuration in shown. Illustration 300 shows a portion of the medical device 100 from a top view perspective, here showing the top jaw 116a and corresponding jaw arm 162a. This configuration in the illustration 300 includes two retractable probes 302 and 304 that can extend beyond the distal end of the jaw 116a, as shown. The probes may be made of shape memory material so as to allow for the probes to be slightly modified in their design or shape, to be used as needed depending on the geometry of the surgical site. A gap 306 exists between the probes 302 and 304, such that the return path of the current may be completed if the ends of the probes 302 and 304 touch tissue. In this case, the second probe 304 acts as the return electrode. In other aspects, a single electrode may be supplied, and a subdermal needle or dispersive electrode in or nearby can act as the return electrode.

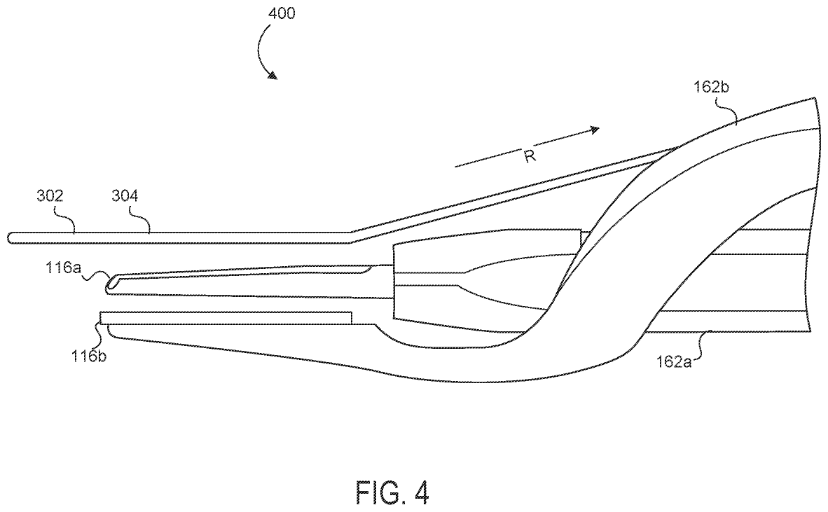

Referring to FIG. 4, illustration 400 shows a side view of the retractable probes 302 and 304, shown in illustration 300. In this configuration, the probes 302 at 304 are located above the jaws 116a and 116b. In other cases, the probes may be located below the jaws or to the sides, and aspects are not so limited. From this view, it may be evident that the probes 302 and 304 may be retracted into the jaw arm 162b in direction R, as shown.

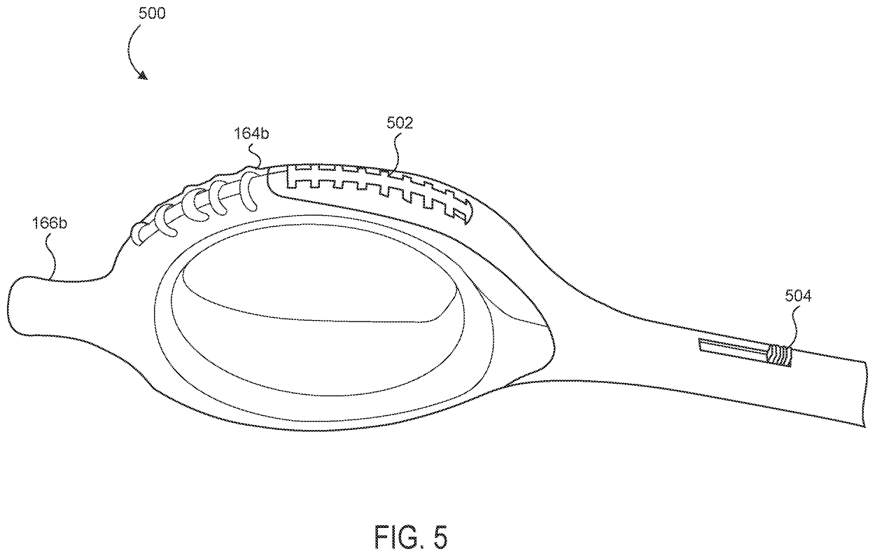

Referring to FIG. 5, illustration 500 shows portions of a retraction mechanism to manipulate the retractable probes, shown in illustrations 300 and 400, according to some aspects. Here, a portion of the jaw handle 164b includes a slot 502 for a slide retraction mechanism to manipulate the retractable probes 302 and 304 (see FIG. 3). In this case, a knob or switch may be configured to slide back and forth within the slot 502 to retract and extend the probes 302 and 304. In other cases, the retractable probes may be manipulated through a wheel retraction mechanism, not shown. Regardless, the jaw handle 164b may include a current amplitude control switch 504 to control the level of current applied to the probes at the end effector.

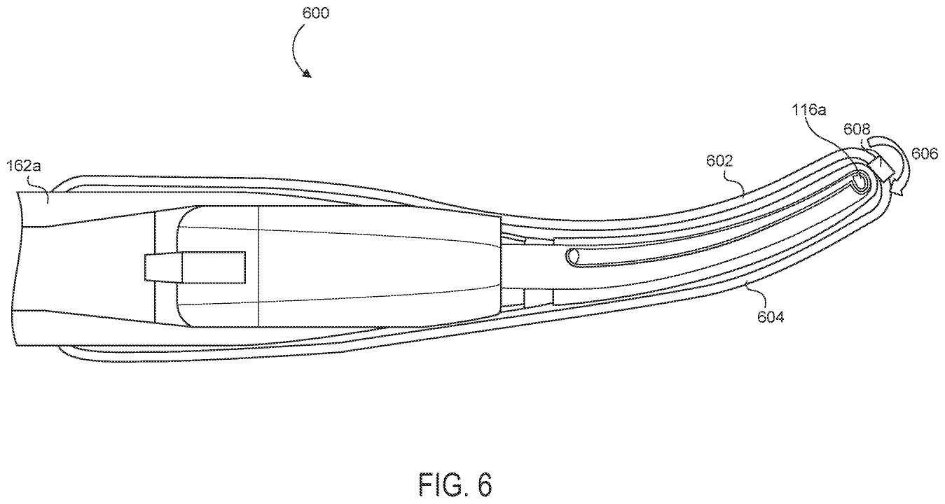

Referring to FIG. 6, illustration 600 shows another alternative design for including a nerve stimulation probe alongside the jaws 116a and 116b, according to some aspects. In this case, probes 602 and 604 are positioned around the perimeter of the jaw 116a and are shaped to follow the contours of the end effector. Illustration 600 shows this configuration from the top view of the medical device. Similar to the configuration in FIG. 3, the probes 602 and 604 are separated by a gap, in this case including a spacer 608 in that gap. Thus, spacer 608 may be secured to the distal end of the jaw 116b so as to stabilize the positions of the probes 602 and 604, or to otherwise prevent movement interference. The current path may again be closed once tissue touches both probes 602 and 604, where the current path travels in the direction of arrow 606. Thus, the probe 604 may act as the return electrode.

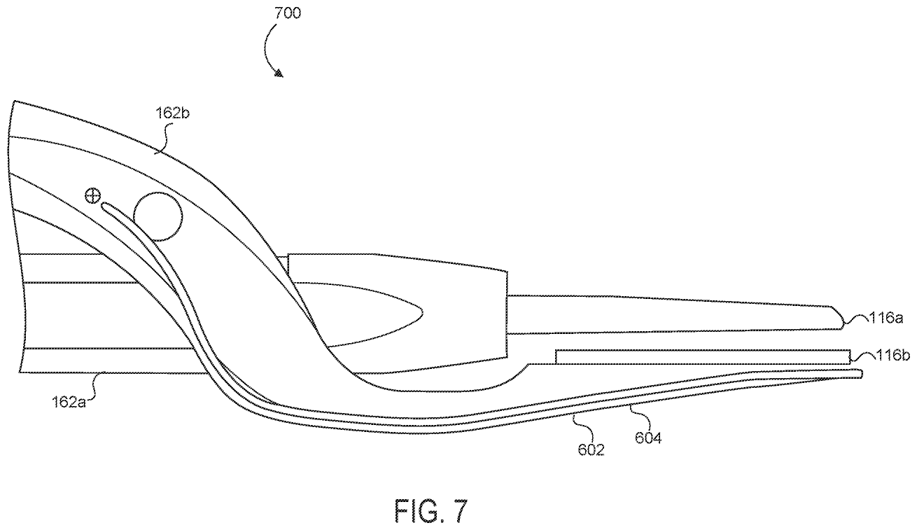

Referring to FIG. 7, illustration 700 shows the side view of the perimeter probes configuration of illustration 600, according to some aspects. Here, the probes 602 and 604 are shown to be positioned on the perimeter of the jaw 116b and a portion of the jaw handle 162b. When combining the perspectives of illustrations 600 and 700, it may be apparent that the probes 602 and 604 follow the contours of the jaw perimeter. In this way, existing medical devices configured to supply therapeutic energy through the jaws may simply be repurposed or retrofitted to include a pair of electrodes or probes running along the perimeter of the jaws, to create a seamless and nonintrusive nerve stimulation feature.

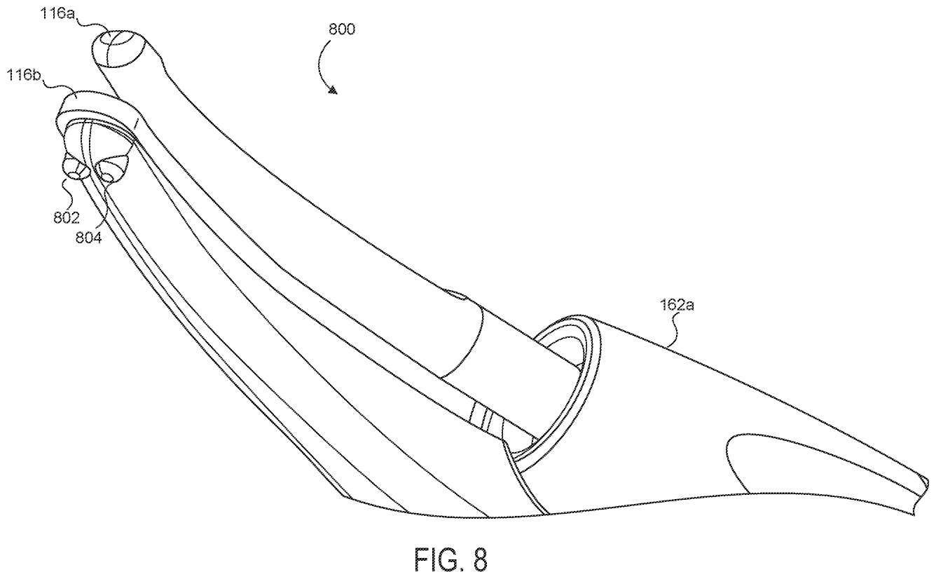

Referring to FIG. 8, illustration 800 shows yet another alternative design for including nerve stimulation at the jaws 116a and 116b, according to some aspects. Here, two small electrode protrusions 802 and 804 at the distal end of the jaw 116b may be used for nerve stimulation and location. One of the protrusions may act as the stimulation probe while the other may act as the return electrode. The circuitry to reach the probes 802 and 804 may run through the jaw down to the distal end of the jaw 116b. While the probes 802 and 804 are shown and positioned on the outside and bottom of the jaw 116b, these probes may be positioned in other configurations. For example, the probes 802 and 804 may be positioned facing the front of the distal end of either jaw 116b or 116a. As another example, the probes 802 and 804 may be positioned on the top of the jaw 116a. In other cases, two sets of probes may be positioned at the distal end of the jaws, one pair on each of the jaws 116a and 116b.

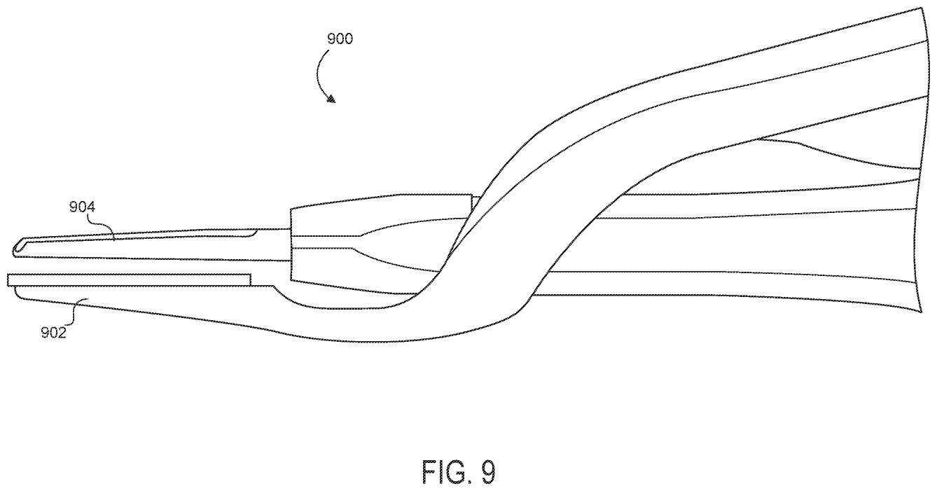

Referring to FIG. 9, illustration 900 shows yet another alternative design for including nerve stimulation in a clamping medical device, according to some aspects. Here, one of the jaws 904 may serve as both a probe and a clamp and/or blade. That is, in one mode of the blade 904, therapeutic energy may be applied to perform various cutting or sealing procedures. In a second mode, the blade 904 may be activated to supply non-therapeutic energy in order to locate and/or stimulate nerves. In some cases, the clamping jaw 902 may act as a return electrode, while in other cases a subdermal needle or a dispersive electrode can act as the return electrode. In addition, it may be apparent that this alternative design in illustration 900 may be combined with the retractable probe, perimeter probe, or tip electrode configurations as discussed in FIGS. 3-8.

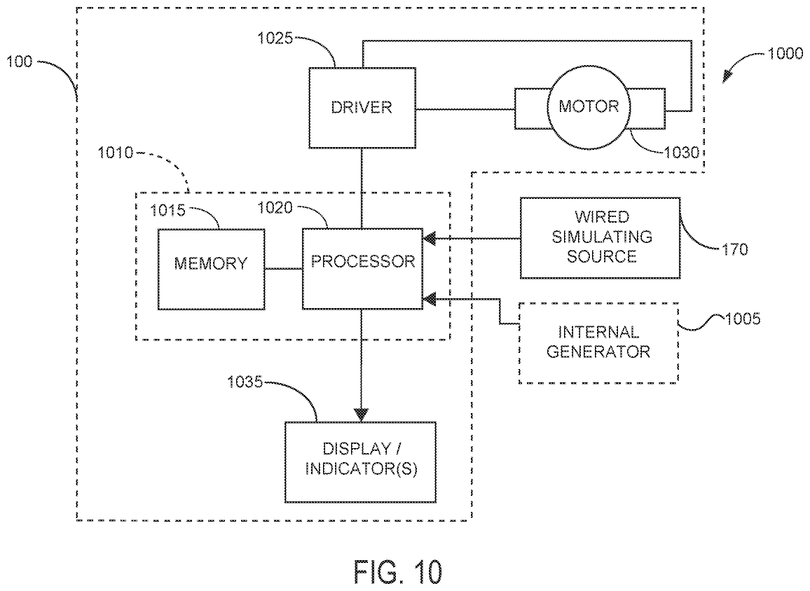

FIG. 10 is a block diagram of a surgical system 1000 comprising a grasping instrument 100 (FIG. 1) with motor-driven components to apply energy to a surgical site, the surgical instrument coupled to a generator wired stimulating source (or alternatively having an internal generator 1005), according to some aspects. The motor 1030 may be employed to close and open either one or both of the jaws 116a, 116b of the end effector 110 (FIG. 2) or firing the knife. Nevertheless, in most aspects, such functions would be hand-operated. The surgical system 1000 shows components of internal circuitry of the grasping instrument, and may also describe the internal circuitry of the alternative grasping instrument 200 (FIG. 2). The motor-driven surgical instrument 100 described in the present disclosure may be coupled to a wired stimulating source 170, such as a generator (or alternatively internal generator 1005) configured to supply power to the surgical instrument through external or internal means. While previous figures describe examples of the structures of the grasping, sealing, cutting, and probing components, FIG. 10 describes examples of the portions for how therapeutic and nontherapeutic energy may be delivered to the end effector. In certain instances, the motor-driven surgical instrument 100 may include a microcontroller 1010 coupled to an external wired generator 170 or internal generator 1005. Either the external generator 170 or the internal generator 1005 may be coupled to A/C mains or may be battery operated or combinations thereof. The electrical and electronic circuit elements associated with the motor-driven surgical instrument 100 and/or the generator elements 170, 1005 may be supported by a control circuit board assembly, for example. The microcontroller 1010 may generally comprise a memory 1015 and a microprocessor 1020 (“processor”) operationally coupled to the memory 1015. The processor 1020 may control a motor driver 1025 circuit generally utilized to control the position and velocity of the motor 1030. The motor 1030 may be configured to control transmission of energy to the electrodes at the end effector of the surgical instrument. In certain instances, the processor 1020 can signal the motor driver 1025 to stop and/or disable the motor 1030, as described in greater detail below. In certain instances, the processor 1020 may control a separate motor override circuit which may comprise a motor override switch that can stop and/or disable the motor 1030 during operation of the surgical instrument in response to an override signal from the processor 1020. It should be understood that the term processor as used herein includes any suitable microprocessor, microcontroller, or other basic computing device that incorporates the functions of a computer's central processing unit (CPU) on an integrated circuit or at most a few integrated circuits. The processor is a multipurpose, programmable device that accepts digital data as input, processes it according to instructions stored in its memory, and provides results as output. It is an example of sequential digital logic, as it has internal memory. Processors operate on numbers and symbols represented in the binary numeral system.

In some cases, the processor 1020 may be any single core or multicore processor such as those known under the trade name ARM Cortex by Texas Instruments. In some cases, any of the surgical instruments of the present disclosures may comprise a safety processor such as, for example, a safety microcontroller platform comprising two microcontroller-based families such as TMS570 and RM4x known under the trade name Hercules ARM Cortex R4, also by Texas Instruments. Nevertheless, other suitable substitutes for microcontrollers and safety processor may be employed, without limitation. In one instance, the safety processor may be configured specifically for IEC 61508 and ISO 26262 safety critical applications, among others, to provide advanced integrated safety features while delivering scalable performance, connectivity, and memory options.

In certain instances, the microcontroller 1010 may be an LM 4F230H5QR, available from Texas Instruments, for example. In at least one example, the Texas Instruments LM4F230H5QR is an ARM Cortex-M4F Processor Core comprising on-chip memory 1015 of 256 KB single-cycle flash memory, or other non-volatile memory, up to 40 MHz, a prefetch buffer to improve performance above 40 MHz, a 32 KB single-cycle serial random access memory (SRAM), internal read-only memory (ROM) loaded with StellarisWare® software, 2 KB electrically erasable programmable read-only memory (EEPROM), one or more pulse width modulation (PWM) modules, one or more quadrature encoder inputs (QEI) analog, one or more 12-bit Analog-to-Digital Converters (ADC) with 12 analog input channels, among other features that are readily available for the product datasheet. Other microcontrollers may be readily substituted for use in the motor-driven surgical instrument 100. Accordingly, the present disclosure should not be limited in this context.

Referring again to FIG. 10, the surgical system 1000 may include a wired stimulating source 170, for example. In certain instances, the wired stimulating source 170 may be configured to supply power through external means, such as through electrical wire coupled to an external generator. In some cases, the surgical system 1000 also may include or alternatively include an internal generator 1005. The internal generator 1005 may be configured to supply power through internal means, such as through battery power or other stored capacitive source. Further descriptions of the internal generator 1005 and the wired generator 170 are described below.

In certain instances, the motor-driven surgical instrument 100 may comprise one or more embedded applications implemented as firmware, software, hardware, or any combination thereof. In certain instances, the motor-driven surgical instrument 100 may comprise various executable modules such as software, programs, data, drivers, and/or application program interfaces (APIs), for example. In certain instances, the surgical instrument 100 may also include one or more displays or other graphical indicators 1035, such as for indicating an energy level being applied to the end effector.

In some aspects, the return electrode may be double purposed to act as the electrical return for both therapeutic and nerve stimulation outputs. In this way, the delivery of the therapeutic energy may be in the form of monopolar electrosurgery, as the single return electrode may obviate the need for bipolar energy delivery. This configuration may be allowed in any of the examples showing a return electrode for the nerve stimulation energy path, including in FIGS. 1-10.

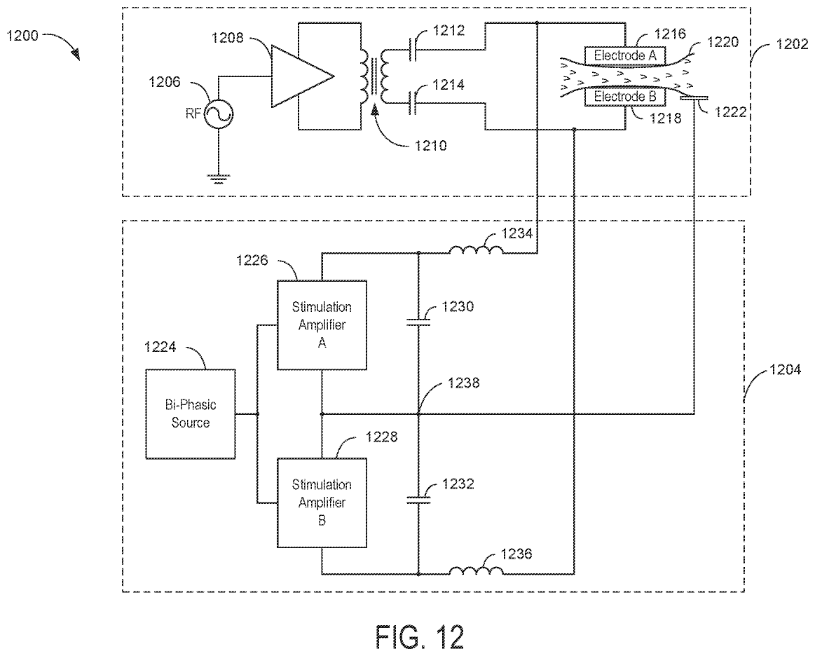

FIG. 12 illustrates one aspect of a nerve stimulation system 1200, according to some aspects of the present disclosure. The nerve stimulation system 1200 provides a technique to stimulate nerves with a bipolar, ultrasonic, or a combination bipolar and ultrasonic instrument with multiple paths. The nerve stimulation system 1200 may represent one example of how the low energy stimulation may be combined with the high energy therapeutic applications in the same medical device, as described in FIGS. 1-11. Typically, a nerve stimulation electrode set is either a pair of closely-spaced electrodes or a probe electrode along with a return pad or needle. Tissue is placed between the first-described pair and stimulated, or the return pad or needle is placed in the appropriate position and then the target tissue is probed with the other electrode. In one aspect, the nerve stimulation system 1200 comprises two separate circuits, A and B, that are driven by the same waveform (preferably a biphasic source, but could be a simple direct current [DC] level). Both circuits A and B have a common return in the pad or needle, but A and B are configured to be out of phase with each other. In the case of a bipolar instrument, the A and B nodes are coupled to the treatment electrodes through an RF blocking circuit (low pass filter) such that the treatment RF is not conducted back through the stimulation circuit or through the needle or pad return of the stimulation circuitry. In this way, tissue can be stimulated between the treatment electrodes through the A and B circuits, and from A to the return or B to the return.

As shown with particularity in FIG. 12, the nerve stimulation system 1200 comprises an RF generator circuit 1202 and a nerve stimulation circuit 1204. The RF generator circuit 1202 comprises an RF source 1206 coupled to an amplifier 1208. The output of the amplifier 1208 is coupled to first and second blocking capacitors 1212, 1214 through an isolation transformer 1210. The first blocking capacitor 1212 is coupled to a first electrode 1216 and the second blocking capacitor 1214 is coupled to a second electrode 1220. As shown, tissue 1220 to be treated is located between the first and second electrodes 126, 1218. An electrically conductive element 1222, such as a pad or needle, is coupled to the tissue 1220 and to a common return 1238 of the nerve stimulation circuit 1204.

Turning now to the nerve stimulation circuit 1204, as shown in FIG. 12, the nerve stimulation circuit 1024 comprises a bi-phasic source 1224 coupled to two separate circuits, a first stimulation amplifier 1226 and a second stimulation amplifier 1228. As shown, the first and second stimulation amplifiers 1226, 1228 are driven by the same waveform, preferably generated by the biphasic source 1224, but they could be driven by a simple DC power source, for example. Both the a first and second stimulation amplifiers 1226, 1228 have a common return 1238 coupled to the tissue 1220 and can be coupled to a pad or needle. The first and second stimulation amplifiers 1226, 1228, however, are configured to be out of phase with each other. In the case of a bipolar instrument, the first and second stimulation amplifiers 1226, 1228 nodes are coupled to the treatment electrodes 1216, 1218 through corresponding RF blocking circuits (e.g., low pass filters), where the first blocking circuit comprises a first capacitor 1230 and a first inductor 1234 and the second blocking circuit comprises a second capacitor 1232 and a second inductor 1236 such that the treatment RF is not conducted back through the stimulation circuit 1204 or through the needle or pad 1222, which is coupled to the common return 1238 of the stimulation circuit 1204. In this way, the tissue 1220 can be stimulated between the treatment electrodes 1216, 1218 through the first and second amplifier circuits 1226, 1228, and from the first amplifier circuit 1226 to the return or from the second amplifier circuit 1228 to the return. Accordingly, first and second poles have separate signal generation circuits comprising first and second amplifier circuits 1226, 1228 with a common return 1238. The path for stimulation can be either through the first amplifier circuit 1226 or through the second amplifier circuit 1228 to the return 1238 as well as from the first amplifier circuit 1226 to the second amplifier circuit 1228 due to the common return 1238.

A combination RF and ultrasonic instrument can be configured to deliver RF energy, ultrasound energy, and can stimulate nerves all in the same instrument/generator. Advantages include, without limitation, simplicity for the surgeon, reduced need for instrument exchange, incorporating the nerve stimulation function within the jaws of the instrument such that tissue can be evaluated for nerves before the full force of grasping is applied to the tissue (avoiding damage to those nerve or structure) and before applying energy destroying the nerves that may be in the grasp of the instrument or treated with the thermal effects of the RF and/or Ultrasound therapeutic energy.

In other aspects, the therapeutic energy treatment may comprise laser, microwave, among other energy sources.

In other aspects, nerve stimulation signals can be generated within the generator either via separate circuits in the handle of the device or with a special output waveform from the generator.

Further, in other aspects, a nerve stimulation signal may be applied between the treatment electrodes automatically prior to the jaws being fully closed so the surgeon can observe any stimulation effect and potentially avoid unwanted nerve damage. In one aspect, a controller may be employed to shut off this function. In another aspect, an indicator may be located on the instrument to show that the nerve stimulation circuit is “on,” e.g., energized, activated, etc., as shown in IEC 60601-2-10, which shows indicators for such stimulation instruments.

In other aspects, a tissue sensing circuit is proved that turns “on,” e.g., energizes, activates, etc., the nerve stimulation signal when tissue is in contact with the stimulation electrodes or the therapeutic electrodes, or ultrasonic blade, or tissue pad.

The energy profile of the nerve stimulation functionality of the present disclosures differs from the energy profiles of the therapeutic functionalities. Aspects of the present disclosures therefore include a description of how nerve stimulation energy may be generated alongside therapeutic energy generation.

In some cases, various aspects may be implemented as an article of manufacture. The article of manufacture may include a computer readable storage medium arranged to store logic, instructions and/or data for performing various operations of one or more aspects. In various aspects, for example, the article of manufacture may comprise a magnetic disk, optical disk, flash memory or firmware containing computer program instructions suitable for execution by a general purpose processor or application specific processor. The aspects, however, are not limited in this context.

The functions of the various functional elements, logical blocks, modules, and circuits elements described in connection with the aspects disclosed herein may be implemented in the general context of computer executable instructions, such as software, control modules, logic, and/or logic modules executed by the processing unit. Generally, software, control modules, logic, and/or logic modules comprise any software element arranged to perform particular operations. Software, control modules, logic, and/or logic modules can comprise routines, programs, objects, components, data structures, and the like that perform particular tasks or implement particular abstract data types. An implementation of the software, control modules, logic, and/or logic modules and techniques may be stored on and/or transmitted across some form of computer-readable media. In this regard, computer-readable media can be any available medium or media useable to store information and accessible by a computing device. Some aspects also may be practiced in distributed computing environments where operations are performed by one or more remote processing devices that are linked through a communications network. In a distributed computing environment, software, control modules, logic, and/or logic modules may be located in both local and remote computer storage media including memory storage devices.

Additionally, it is to be appreciated that the aspects described herein illustrate example implementations, and that the functional elements, logical blocks, modules, and circuits elements may be implemented in various other ways which are consistent with the described aspects. Furthermore, the operations performed by such functional elements, logical blocks, modules, and circuits elements may be combined and/or separated for a given implementation and may be performed by a greater number or fewer number of components or modules. As will be apparent to those of skill in the art upon reading the present disclosure, each of the individual aspects described and illustrated herein has discrete components and features which may be readily separated from or combined with the features of any of the other several aspects without departing from the scope of the present disclosure. Any recited method can be carried out in the order of events recited or in any other order which is logically possible.

Unless specifically stated otherwise, it may be appreciated that terms such as “processing,” “computing,” “calculating,” “determining,” or the like, refer to the action and/or processes of a computer or computing system, or similar electronic computing device, such as a general purpose processor, a DSP, ASIC, FPGA, or other programmable logic device, discrete gate or transistor logic, discrete hardware components, or any combination thereof designed to perform the functions described herein that manipulates and/or transforms data represented as physical quantities (e.g., electronic) within registers and/or memories into other data similarly represented as physical quantities within the memories, registers, or other such information storage, transmission, or display devices.

It is worthy to note that some aspects may be described using the expression “coupled” and “connected” along with their derivatives. These terms are not intended as synonyms for each other. For example, some aspects may be described using the terms “connected” and/or “coupled” to indicate that two or more elements are in direct physical or electrical contact with each other. The term “coupled,” however, also may mean that two or more elements are not in direct contact with each other, but yet still co-operate or interact with each other. With respect to software elements, for example, the term “coupled” may refer to interfaces, message interfaces, and application program interface, exchanging messages, and so forth.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Although various aspects have been described herein, many modifications, variations, substitutions, changes, and equivalents to those aspects may be implemented and will occur to those skilled in the art. Also, where materials are disclosed for certain components, other materials may be used. It is therefore to be understood that the foregoing description and the appended claims are intended to cover all such modifications and variations as falling within the scope of the disclosed aspects. The following claims are intended to cover all such modification and variations.

1. A surgical instrument comprising:
a first jaw handle and a second jaw handle pivotally coupled about a joint;
the first jaw handle comprising a first jaw at a distal end of the first jaw handle;
the second jaw handle comprising a second jaw at a distal end of the second jaw handle;
the first and second jaws configured to grasp tissue therebetween when the first and second jaw handles are pivotally rotated about the joint into a closed position;
at least one of the first and second jaws configured to deliver therapeutic energy at a higher energy level for sealing or cutting tissue at a surgical site; and
at least one of the first and second jaws configured to deliver nontherapeutic energy at a lower energy level for providing electrical stimulation to nerve tissue at the surgical site.

2. The surgical instrument of claim 1, wherein the therapeutic energy comprises ultrasonic energy.

3. The surgical instrument of claim 1, wherein the therapeutic energy comprises high frequency electrosurgical energy.

4. The surgical instrument of claim 1, wherein both the first and second jaws are configured to deliver the nontherapeutic energy to the tissue at the surgical site, and wherein the nontherapeutic energy delivered at both the first and second jaws are at the same electrical potential.

5. The surgical instrument of claim 1, wherein at least one of the first and second jaws comprises an insulating material on an inside portion of said jaw such that the first jaw contacts the second jaw in the closed position by way of touching the insulating material.

6. The surgical instrument of claim 1, wherein the first jaw arm and the second jar arm are pivotally coupled at a joint.

7. A method of operating a surgical instrument, the method comprising:
moving a first jaw coupled to a distal end of a first jaw arm and a second jaw coupled to a distal end of a second jaw arm of the surgical instrument from an open configuration to a closed configuration to capture tissue positioned between the first jaw and the second jaw;
energizing an electrode of the surgical instrument to deliver a first energy to tissue positioned between the first jaw and the second jaw; and
moving a probe of the surgical instrument from a retracted configuration in which the probe is retracted into a housing of the first jaw arm or a housing of the second jaw arm to an extended configuration; and
energizing the probe to deliver a second energy to the tissue.

8. The method of claim 7, wherein the second energy comprises nontherapeutic energy for providing electrical stimulation to the tissue

9. A nerve stimulation system of a surgical instrument, the nerve stimulation system comprising:
an RF generator circuit including a first electrode and a second electrode; and
an nerve stimulation circuit including a bi-phasic source, a first amplifier, and a second amplifier.

10. The nerve stimulation system of claim 9, wherein the first amplifier and the second amplifier are out of phase relative to each other.
